(19) United States
(12) Patent Application Publication
(10) Pub. No.: US 2023/0320657 A1
(43) Pub. Date: Oct. 12, 2023

(54) DEVICES, METHODS AND PROGRAMS FOR MANAGING PATIENTS WITH RESPIRATORY DISEASE

(71) Applicant: THE CATHOLIC UNIVERSITY OF KOREA INDUSTRY-ACADEMIC COOPERATION FOUNDATION, Seoul (KR)

(72) Inventor: Hwa Young LEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/297,508

(22) Filed: Apr. 7, 2023

(30) Foreign Application Priority Data
Apr. 8, 2022 (KR) 10-2022-0043864

(51) Int. Cl. A61B 5/00; A61B 5/087; G16H 10/60

(52) U.S. Cl. CPC A61B 5/4848; A61B 5/7267; A61B 5/087; A61B 2560/0242; A61B 5/0004; A61B 5/743; G16H 10/60; A61B 5/7275

(57) ABSTRACT

The inventive concept relates to a respiratory disease patient management device, and may determine a patient's respiratory state based on the patient's peak expiratory flow and asthma symptom score, and may provide information about the additional dose of medication required by the patient by determining the patient's respiratory state based on the patient's peak expiratory flow and asthma symptom score.

### CROSS-REFERENCE TO RELATED APPLICATIONS

A claim for priority under 35 U.S.C. § 119 is made to Korean Patent Application No. 10-2022-0043864 filed on Apr. 8, 2022 in the Korean Intellectual Property Office, the entire contents of which are hereby incorporated by reference.

### BACKGROUND

Embodiments of the inventive concept described herein relate to a device for managing patients with respiratory diseases.

Nowadays, respiratory volume management and therapeutic aids/devices for patients with respiratory diseases are being actively developed, but need to go to the hospital to use it.

Moreover, as the concentration of fine dust increases, the number of patients with various respiratory diseases has recently increased. However, there is no way to effectively educate patients on a method of these patients managing their respiratory disease alone and to practice the method.

Furthermore, because treatment and assistance for patients with respiratory diseases are performed in only the hospital, there are difficulties in collecting relevant data for research on patients with respiratory diseases.

### SUMMARY

Embodiments of the inventive concept provide a device for managing a patient with a respiratory disease that allows the patient with a respiratory disease to manage the respiratory disease by himself/herself.

Embodiments of the inventive concept determine the patient's respiratory state based on the patient's peak expiratory flow and asthma symptom score.

Embodiments of the inventive concept provide information about the additional amount of medication required for the patient by determining the patient's respiratory state based on the patient's peak expiratory flow and asthma symptom score.

Problems to be solved by the inventive concept are not limited to the problems mentioned above, and other problems not mentioned will be clearly understood by those skilled in the art from the following description.

According to an embodiment, a respiratory disease patient management device includes an artificial intelligence (AI) model that determines a respiratory state of a patient based on a peak expiratory flow and asthma symptom score of the patient, a communication unit that communicates with a patient terminal, and at least one processor. The processor receives, from the patient terminal, asthma medication intake status of the patient and the peak expiratory flow (PEF) of the patient, calculates a first asthma symptom score of the patient based on a value entered for a plurality of asthma symptom questions provided to the patient terminal, calculates first state information about the patient by inputting the received peak expiratory flow and the calculated first asthma symptom score into the AI model, and generates respiratory disease management information including whether the patient needs additional medication, based on the calculated first state information when at least one symptom information about the patient is received from the patient terminal, and transmits the generated respiratory disease management information to the patient terminal such that information for respiratory disease management of the patient is displayed on the patient terminal.

According to an embodiment, a method performed by a respiratory disease patient management device includes receiving, from a patient terminal, asthma medication intake status of the patient and a peak expiratory flow (PEF) of the patient, receiving a value entered for a plurality of asthma symptom questions provided to the patient terminal, calculating a first asthma symptom score of the patient based on the received value, when at least one symptom information about the patient is received from the patient terminal, calculating first state information about the patient by inputting the received peak expiratory flow and the calculated first asthma symptom score into an AI model, generating respiratory disease management information including whether the patient needs additional medication, based on the calculated first state information, and transmitting the generated respiratory disease management information to the patient terminal and displaying information for respiratory disease management of the patient on the patient terminal. The AI model determines a respiratory state of the patient based on the peak expiratory flow of the patient and an asthma symptom score of the patient.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIGS. 1 and 2 are block diagrams of a respiratory disease patient management system, according to an embodiment of the inventive concept;

FIG. 3 is a flowchart of a respiratory disease patient managing method, according to an embodiment of the inventive concept;

FIG. 4 is a diagram illustrating that an artificial intelligence model calculates a patient's first asthma symptom score;

FIG. 5 is a diagram illustrating providing daily data for a patient's peak expiratory flow;

FIG. 6 is a diagram illustrating a request for a patient's input of medication information;

FIG. 7 is a diagram illustrating a request for basic information input including a patient's height and basic lung capacity;

FIGS. 8 to 12 are diagrams illustrating providing a peak expiratory flow measuring method;

FIG. 13 is a diagram illustrating at least part of a plurality of asthma symptom questions provided to patients;

FIG. 14 is a diagram illustrating providing information about a patient's respiratory state today, selecting a symptom of the day, and requesting for entering a cause when there is the selected symptom;

FIG. 15 is a diagram illustrating a function of requesting the determination as to whether a patient needs additional use of medication;

FIG. 16 is a diagram illustrating an algorithm for determining whether a patient additionally uses medication;

FIG. 17 is a diagram illustrating providing information about the patient's use history of an inhaler;

FIG. 18 is a diagram illustrating providing information about a patient's asthma symptom score history; and,

FIG. 19 is a diagram illustrating providing information about a patient's symptom record history.

### DETAILED DESCRIPTION

The above and other aspects, features and advantages of the inventive concept will become apparent from the following description of the following embodiments given in conjunction with the accompanying drawings. The inventive concept, however, may be embodied in various different forms, and should not be construed as being limited only to the illustrated embodiments. Rather, these embodiments are provided as examples so that the inventive concept will be thorough and complete, and will fully convey the scope of the inventive concept to those skilled in the art. The inventive concept may be defined by the scope of the claims.

The terms used herein are provided to describe embodiments, not intended to limit the inventive concept. In the specification, the singular forms include plural forms unless particularly mentioned. The terms “comprises” and/or “comprising” used herein do not exclude the presence or addition of one or more other components, in addition to the aforementioned components. The same reference numerals denote the same components throughout the specification. As used herein, the term “and/or” includes each of the associated components and all combinations of one or more of the associated components. It will be understood that, although the terms “first”, “second”, etc., may be used herein to describe various components, these components should not be limited by these terms. These terms are only used to distinguish one component from another component. Thus, a first component that is discussed below could be termed a second component without departing from the technical idea of the inventive concept.

Unless otherwise defined, all terms (including technical and scientific terms) used herein have the same meaning as commonly understood by those skilled in the art to which the inventive concept pertains. The terms, such as those defined in commonly used dictionaries, should not be interpreted in an idealized or overly formal sense unless expressly so defined herein.

Hereinafter, an embodiment of the inventive concept will be described in detail with reference to the accompanying drawings.

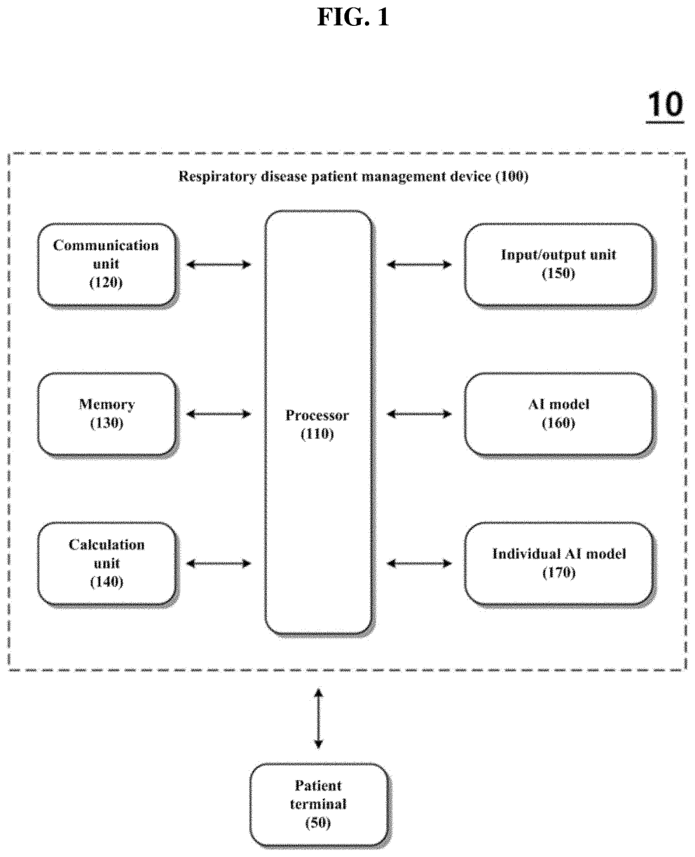

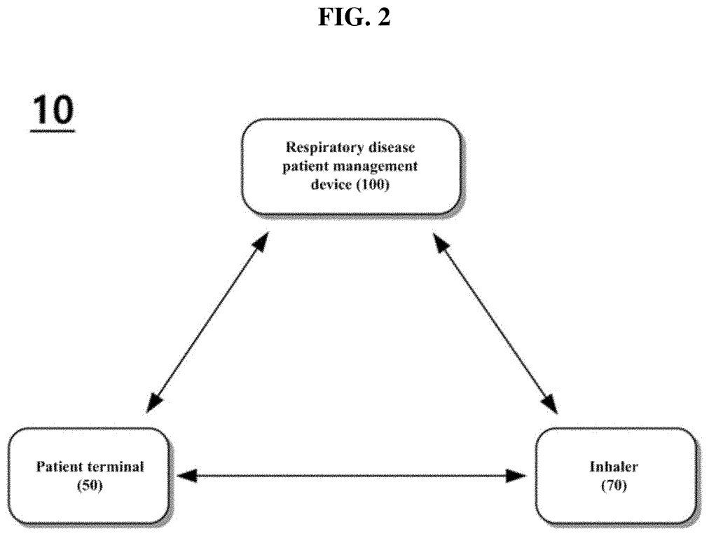

FIGS. 1 and 2 are block diagrams of a respiratory disease patient management system 10, according to an embodiment of the inventive concept.

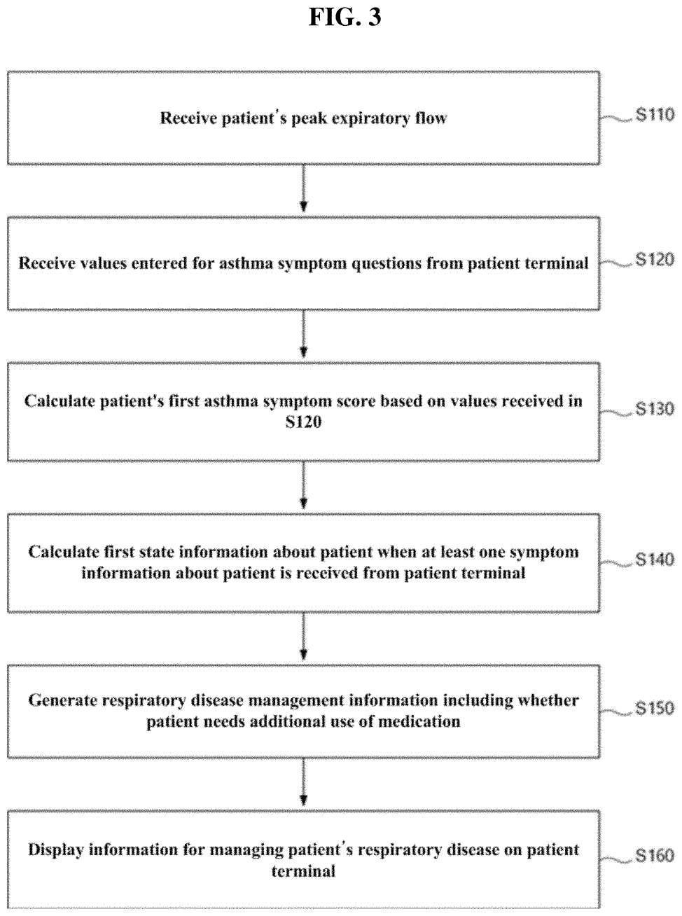

FIG. 3 is a flowchart of a respiratory disease patient managing method, according to an embodiment of the inventive concept.

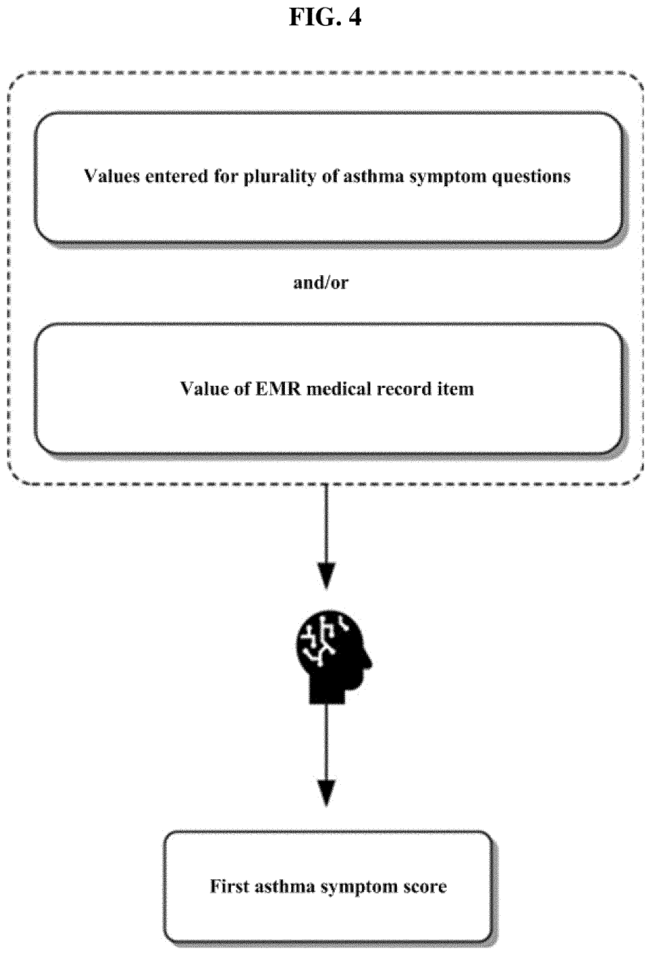

FIG. 4 is a diagram illustrating that an artificial intelligence model calculates a patient's first asthma symptom score.

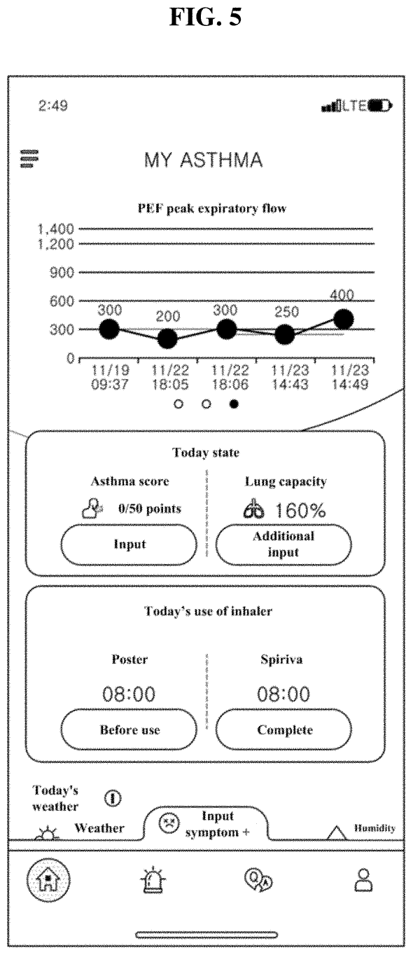

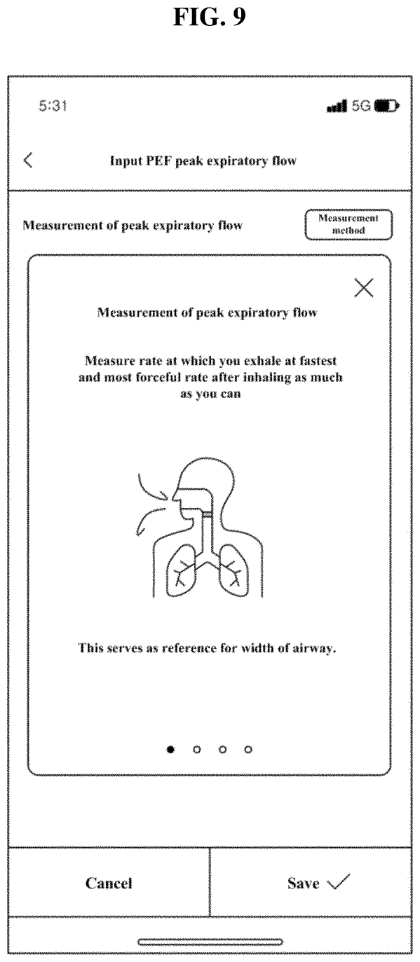

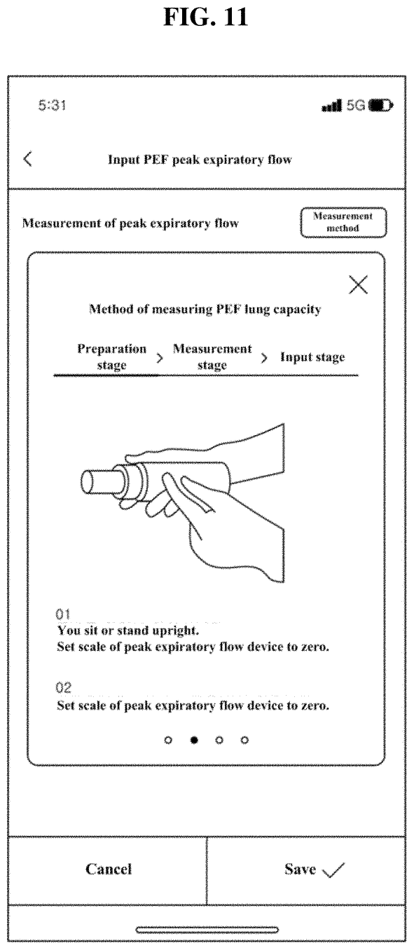

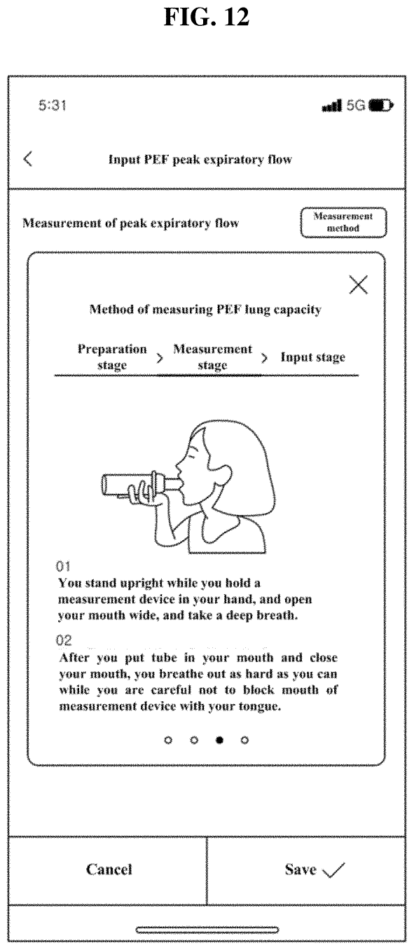

FIGS. 5 to 18 are various views for describing a device 100 and method for managing patients with a respiratory disease, according to an embodiment of the inventive concept.

Hereinafter, the device 100, method, and program for managing patients with a respiratory disease, according to an embodiment of the inventive concept will be described with reference to FIGS. 1 and 2, and will be described with reference to FIGS. 5 to 18 as needed.

Referring to FIG. 1, the respiratory disease patient management system 10 according to an embodiment of the inventive concept includes the respiratory disease patient management device 100 and a patient terminal 50.

Moreover, the respiratory disease patient management device 100 includes a processor 110, a communication unit 120, a memory 130, a calculation unit 140, an input/output unit 150, an artificial intelligence (AI) model 160, and an individual AI model 170.

However, in some embodiments, a server may include fewer or more components than the components illustrated in FIG. 1.

Moreover, referring to FIG. 2, the respiratory disease patient management system 10 according to an embodiment of the inventive concept may further include an inhaler 70.

The inhaler 70 may include a sensor. The inhaler 70 may generate inhalation sensing data through the sensor and may transmit the inhalation sensing data to the patient terminal 50 through a communication unit.

Accordingly, the patient terminal 50 may receive the inhalation sensing data according to the patient's use of the inhaler 70 from the inhaler 70 by communicating with the inhaler 70 and may count the number of uses of the inhaler 70 of the patient and the number of remaining uses of the inhaler 70 based on the inhalation sensing data.

This will be more fully described later.

The communication unit 120 includes a wired/wireless communication module and communicates with the patient terminal 50.

The memory 130 may store various commands, algorithms, and programs for executing the respiratory disease patient management method according to an embodiment of the inventive concept, and may store the patient's respiratory disease patient management service use history.

In detail, the memory 130 may store at least one piece of information of the patient's peak expiratory flow input history, the patient's asthma medication intake history, a first asthma symptom score history calculated for the patient, a plurality of asthma symptom questions to be delivered to the patient terminal 50, and cause information about the patient's symptom.

According to an embodiment of the inventive concept, the respiratory disease patient management device 100 according to an embodiment of the inventive concept may store various types of information in the memory 130, may generate the stored information as a learning dataset to train the AI model 160, and may provide effects capable of being utilized as big data.

Under control of the processor 110, the calculation unit 140 may calculate a first asthma symptom score, a second asthma symptom score, and the like.

The input/output unit 150 may receive various types of information, data, and input signals, and may output various types of generated information.

The artificial intelligence model 160 may determine the patient's respiratory disease status based on the patient's peak expiratory flow and asthma symptom score.

The artificial intelligence model 160 is built by inputting learning data generated by using a peak expiratory flow measured for a plurality of patients, asthma symptom scores for various cases, the patient's respiratory disease state information, and each patient's surrounding environment information.

In detail, the processor 110 may generate the learning dataset by using at least one of the patient's peak expiratory flow collected from a plurality of patients, the patient's asthma symptom score, the patient's respiratory disease state information, the patient's symptom, the patient's cause information about the symptom, the calculated first asthma symptom score of the patient, and the patient's surrounding environment information and may enter the generated learning dataset to an AI model for training.

The individual AI model 170 is based on the AI model 160, and is a model learned by using data of each patient to accurately identify a state of each patient.

In the above-described embodiment, it is described that the processor 110 calculates the first asthma symptom score based on a value entered for a plurality of asthma symptom questions provided to a patient terminal, but is not limited thereto.

Referring to FIG. 4, it is described that an artificial intelligence model calculates the patient's first asthma symptom score.

In detail, the processor 110 may load the patient's EMR medical record, and may calculate the first asthma symptom score based on at least one of the patient's EMR medical record and values entered for a plurality of asthma symptom questions provided to the patient terminal.

Accordingly, the processor 110 may calculate the first asthma symptom score by using the patient's EMR medical record or the values entered for a plurality of asthma symptom questions provided to the patient terminal, or may calculate the first asthma symptom score by using both the patient's EMR medical record and the values.

At this time, the processor 110 may use at least one of allergy information, body fat index, medical questionnaire directly measured at the hospital, smoking history, and underlying diseases (e.g., rhinitis, etc.) related to respiratory organs from the patient's EMR medical record.

At this time, the respiratory disease patient management device 100 may receive an EMR medical record from the patient terminal 50 or a medical staff client device, or may load the EMR medical record by accessing a DB in which the EMR medical record is stored.

The AI Model 160 may provide AI guidance to the patient terminal.

Besides, the AI model 160 may provide guidance by using an instructional algorithm. The instructional algorithm may be learned through a method such as reinforcement learning, imitation learning, or the like, and may operate on a rule basis.

As described above, the memory 130 stores at least one rule for the AI model 160 to operate.

The processor 110 may be in charge of controlling components within the respiratory disease patient management device 100 and may execute a respiratory disease patient management method by using various commands, algorithms and models.

As an example, the processor 110 may obtain location information of the patient terminal 50 by activating a GPS function of the patient terminal 50.

When the location information is obtained from the patient terminal 50, the processor 110 collects surrounding environment information at a location corresponding to the obtained location information.

The periphery from the patient terminal means at least one preset distance range from the patient terminal. For example, 100 m, 200 m, 500 m, 1 Km, or the like is capable of being applied to the periphery.

The surrounding environment information may be applied to anything related to the environment, such as allergy information, temperature, humidity, fine dust concentration, and meteorological information that are capable of occurring at the corresponding location.

The processor 110 may store, the memory 110, the patient's peak expiratory flow input history, the patient's asthma medication intake history, a first asthma symptom score history calculated for the patient, a plurality of asthma symptom questions to be delivered to the patient terminal 50, cause information about the patient's symptom, and the surrounding environment information.

Furthermore, the processor 110 may generate a learning dataset by using at least one of the patient's peak expiratory flow input history stored and accumulated in a memory at each preset cycle, the patient's asthma medication intake history, the first asthma symptom score history calculated for the patient, the plurality of asthma symptom questions to be provided to the patient terminal 50, the cause information about the patient's symptom, and the surrounding environment information. The processor 110 may build a new artificial intelligence model, or may update an existing artificial intelligence model by entering the generated learning dataset into an artificial intelligence model for training.

The processor 110 may provide respiratory management information to the patient by using at least one of date information, season information, and weather information through the individual AI model 170.

For example, the processor 110 may provide the patient terminal 50 with information indicating paying attention to a respiratory system and wearing a mask because the fine dust index is high at a period in which a level of fine dust is high.

For example, when pollen is blown in spring and a patient has pollen allergy, the processor 110 may provide the patient terminal 50 with pollen information and allergy warning information by using the individual AI model 170.

An operation of the respiratory disease patient management device 100 will be described in detail with reference to FIG. 2.

The respiratory disease management device 100 according to an embodiment of the inventive concept may include a server device, and may provide a respiratory disease management service to the patient terminal 50 through a service application and web.

FIG. 5 is a diagram illustrating providing daily data for a patient's peak expiratory flow.

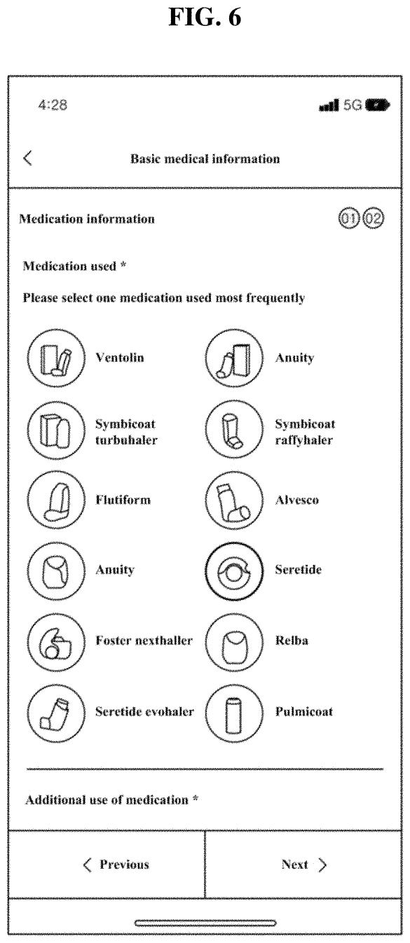

FIG. 6 is a diagram illustrating a request for a patient's input of medication information.

When a health management mode is executed through the service application of the patient terminal 50, the processor 110 may provide the patient terminal 50 with daily data on a patient's peak expiratory flow, an asthma score, the patient's today state information including lung capacity, and today use of the inhaler 70, as illustrated in FIG. 5.

The processor 110 requests the patient to enter basic medical information of the patient through the patient terminal 50 and requests the patient to select an asthma medication currently being taken as illustrated in FIG. 6.

When asthma medication is selected from the patient terminal 50, the processor 110 may request the patient terminal 50 to enter the number of times of medication use and the time of use. The processor 110 may store this information in the memory 130 and may generate a push notification signal for providing a notification of medication intake according to the medication intake time set by the patient.

Moreover, when the medication intake or an intake time is changed by the patient terminal 50, the processor 110 may store and apply the changed settings to the memory 130.

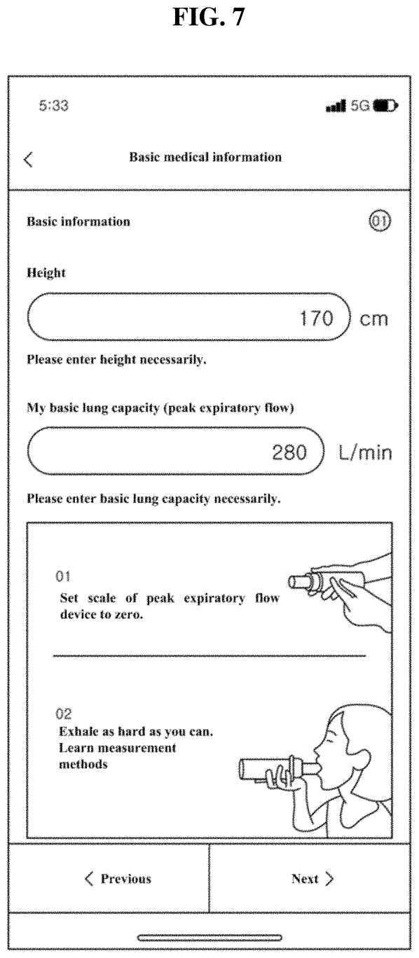

FIG. 7 is a diagram illustrating a request for basic information input including a patient's height and basic lung capacity.

Referring to FIG. 7, the processor 110 request a patient to enter the patient's height and basic lung capacity (peak expiratory flow) as the patient's basic medical information, and stores the information entered through the patient terminal 50 in the memory 130.

In an embodiment, the processor 110 may provide at least one individual evaluation item to the patient terminal 50, and may request the patient to check or enter a value for an item having the relation. The individual evaluation item may include at least one of cold, fine dust, house dust, pets, mold, pollen, cold air, humidity, transportation, smell, smoking, and stress. The processor 110 may store the received information in the memory 130.

The processor 110 may analyze information received from the patient terminal 50 for the items by using the AI model 160 or the individual AI model 170, may derive suggestion information capable of improving the patient's respiratory state, and may provide the suggestion information to the patient terminal 50.

For example, the processor 110 may provide suggestion information, for example, “please cut the pet with the patient terminal 50.”, “please spend less time with your pet.”, “please lower the humidity by XX.”, or the like.

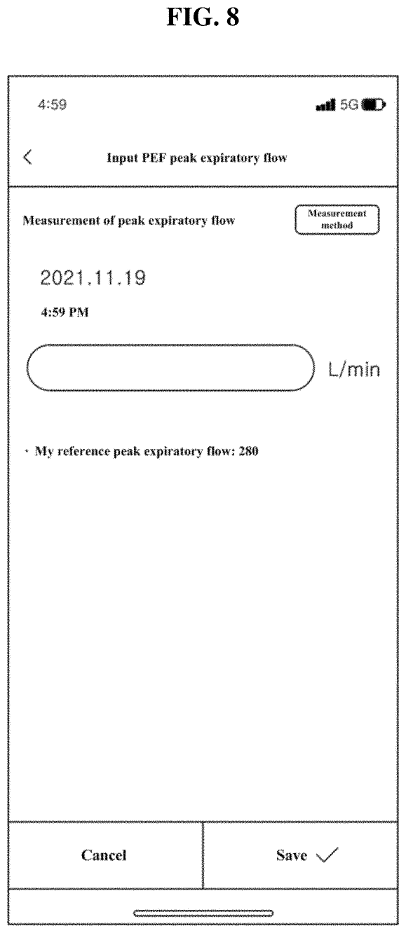

FIGS. 8 to 12 are diagrams illustrating providing a peak expiratory flow measuring method.

When a peak expiratory flow measuring method is requested from the patient terminal 50, the processor 110 may provide the patient terminal 50 with guide information stored in the memory 130 as shown in FIGS. 8 to 12.

The respiratory disease patient management device 100 receives a patient's peak expiratory flow (PEF) through the communication unit 120 (S110).

The processor 110 provides a plurality of asthma symptom questions to the patient terminal 50 so as to be output to the patient terminal 50.

The processor 110 receives values entered for the asthma symptom questions from the patient terminal 50 through the communication unit 120 (S120).

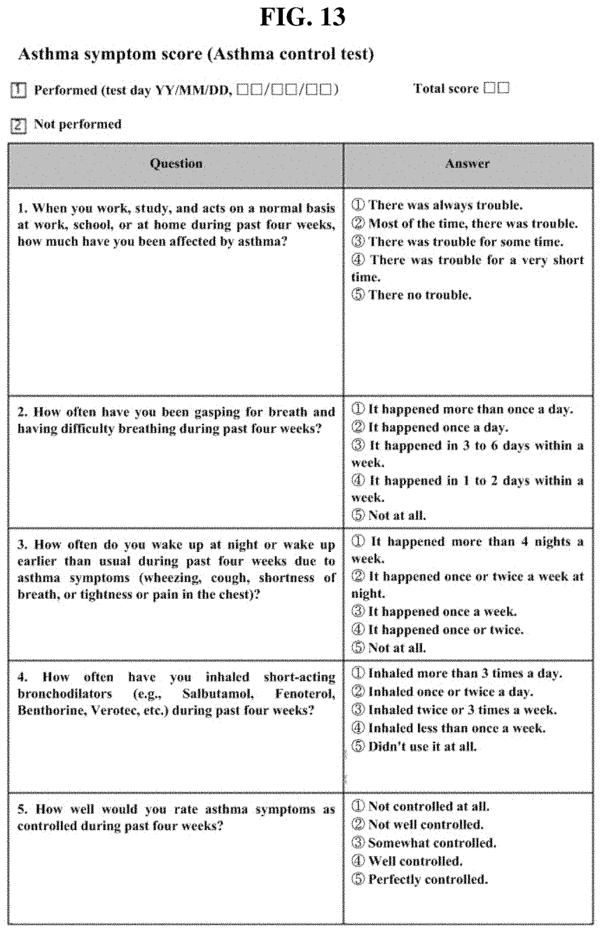

FIG. 13 is a diagram illustrating at least part of a plurality of asthma symptom questions provided to patients.

The processor 110 provides the patient terminal 50 with a plurality of asthma symptom questions as shown in FIG. 13, and requests a patient to enter the patient's today (current) state.

The processor 110 calculates the patient's first asthma symptom score based on the values received in S120 (S130).

When at least one symptom information about the patient is received from the patient terminal 50, the processor 110 calculates first state information about the patient (S140).

The processor 110 generates respiratory disease management information including whether the patient needs additional use of medication (S150).

The processor 110 displays information about the patient's respiratory disease management on the patient terminal 50 (S160).

In detail, in S160, the processor 110 may transmit the respiratory disease management information generated in S150 to the patient terminal 50 such that information for managing the patient's respiratory disease is displayed on the patient terminal 50.

The processor 110 may calculate the patient's first asthma symptom score based on the values for the plurality of asthma symptom questions received in S120. In detail, as illustrated in FIG. 13, each asthma symptom question may have multiple different answers. The processor 110 may calculate the patient's first asthma symptom score depending on the type of a question and the type of an answer.

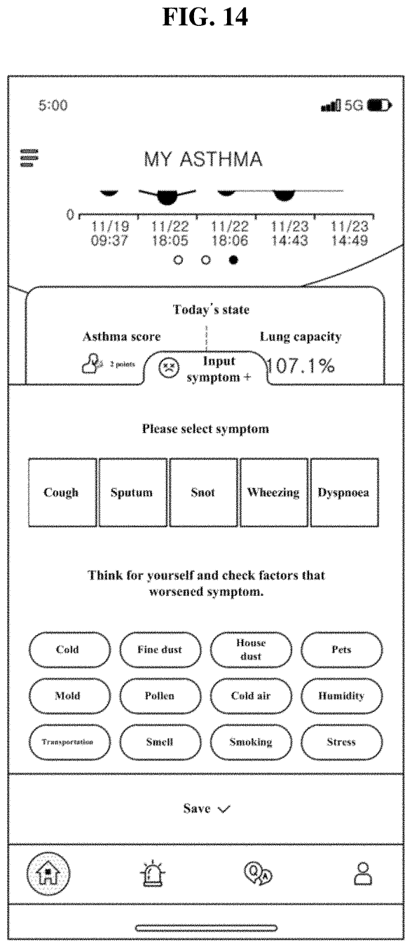

FIG. 14 is a diagram illustrating providing information about a patient's respiratory state today, selecting a symptom of the day, and requesting for entering a cause when there is the selected symptom.

Referring to FIG. 14, the processor 110 may provide the patient terminal 50 with today state information including the patient's asthma score and lung capacity.

Moreover, the processor 110 requests a patient to select at least one symptom, which is currently experienced, through the patient terminal 50. When the symptom is selected, the processor 110 requests the patient to select a cause (factor) of the symptom that the patient thinks.

Accordingly, the processor 110 may receive at least one symptom, which the patient is currently experiencing, from the patient terminal 50 and may receive a cause (factor) of at least one symptom received from the patient terminal 50.

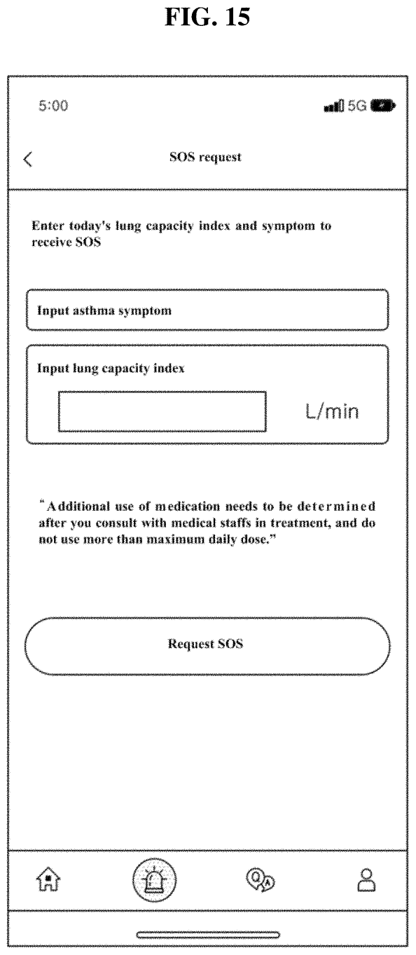

FIG. 15 is a diagram illustrating a function of requesting the determination as to whether a patient needs additional use of medication.

Referring to FIG. 15, when at least one symptom information about a patient is received from the patient terminal 50 or a signal (SOS request signal) for determining additional use of medication is received, the processor 110 may perform the following process.

However, it is not limited to this, and in some embodiments, the respiratory disease patient management device 100 may calculate a score for the patient's current respiratory state by inputting a peak expiratory flow and first asthma symptom score to the AI model 160. When it is determined based on the score that the patient's respiratory state is not good, the respiratory disease patient management device 100 may automatically proceed with the following process.

At this time, the processor 110 may provide the patient terminal 50 with precautions according to the use of the additional medication use algorithm as shown in FIG. 15.

The processor 110 may calculate first state information about the patient by inputting the peak expiratory flow received from the patient terminal 50 and the calculated first asthma symptom score to the AI model 160.

At this time, the AI model 160 compares the peak expiratory flow received from the patient terminal 50 with a reference peak expiratory flow and compares the calculated first asthma symptom score with the first reference score.

Afterward, the AI model 160 calculates first state information about the patient based on the comparison result.

In this case, the reference peak expiratory flow and first reference score may be preset to determine additional use of medication for respiratory disease patients. In some embodiments, different values may be set for each patient.

The processor 110 generates respiratory disease management information including whether the patient needs additional use of medication based on the calculated first state information.

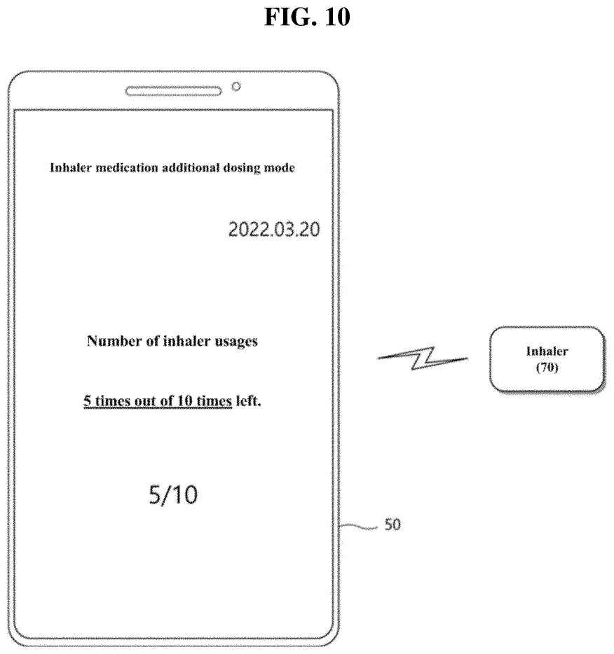

FIG. 10 is a diagram illustrating that the processor 110 guides the number of uses of the inhaler 70 through a patient terminal.

In an embodiment, when a patient needs the additional use of medication, the processor 110 may instruct the number of uses of the inhaler 70 for inhaling medication.

In detail, the processor 110 may provide a notification of the number of times that a patient inhales medication, by using the AI model 160. The patient may inhale the correct amount of medication by using an inhaler the number of times.

In an embodiment, the inhaler 70 may include a sensor and a communication unit. The sensor may sense the patient's use (inhalation) of the inhaler 70.

The patient terminal 50 may be paired with the inhaler 70 through the communication unit, and may receive usage sensing data (inhalation sensing data) of the patient's inhaler 70 from the sensor of the inhaler 70.

The patient terminal 50 may transmit usage sensing data of the inhaler 70 to the respiratory disease patient management device 100 through the communication unit.

The patient terminal 50 or the respiratory disease patient management device 100 may count the number of uses of the inhaler 70 based on the usage sensing data of the patient's inhaler 70 and may output the number of remaining usages to the patient terminal 50.

In detail, the patient terminal 50 or the respiratory disease patient management device 100 may calculate the number of times that the patient uses the inhaler 70, based on respiratory disease management information indicating whether the patient needs additional medication and may provide the patient terminal 50 with the number of times that the patient uses the inhaler 70. In addition, the patient terminal 50 or the respiratory disease patient management device 100 may count the number of remaining usages based on the sensing data of the inhaler 70 and may output the counting result to the patient terminal 50, thereby allowing the patient to inhale a precise amount of medication.

At this time, the exact amount means the number of additional usages of medication calculated based on the patient's state information.

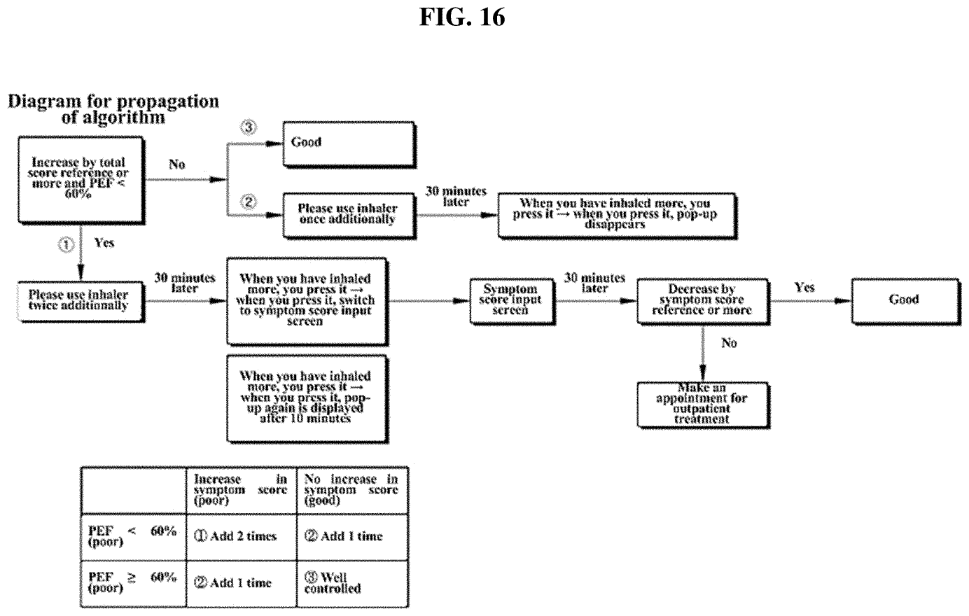

FIG. 16 is a diagram illustrating an algorithm for determining whether a patient additionally uses medication.

FIG. 16 illustrates an algorithm for determining a patient's additional use of medication, which is used by the respiratory disease patient management device 100 according to an embodiment of the inventive concept.

In an embodiment, when a peak expiratory flow received from the patient terminal 50 is greater than or equal to a reference peak expiratory flow, and the first asthma symptom score is increased to be greater than the first asthma symptom score on a previous test by a predetermined score or more, or when the first asthma symptom score does not exceed the first asthma symptom score on the previous test and the peak expiratory flow received from the patient terminal 50 is less than the reference peak expiratory flow, the AI model 160 may determine that the patient needs one additional dose of medication.

In an embodiment, when the peak expiratory flow received from the patient terminal 50 is less than the reference peak expiratory flow, and the first asthma symptom score is greater than or equal to the first reference score, the AI model 160 may determine that the patient needs two or more additional doses of medication.

Next, when the patient completes 2 additional doses of medication, after a predetermined time, the processor 110 provides again the plurality of asthma symptom questions, which are provided to the patient terminal 50, and calculates the patient's second asthma symptom score based on values entered for a plurality of asthma symptom questions thus provided again.

Besides, the processor 110 may calculate second state information about the patient based on a second asthma symptom score and may determine whether the patient needs an outpatient appointment, based on the calculated second state information.

In detail, when the second asthma symptom score is decreased to be less than the first asthma symptom score by a predetermined score or more, the processor 110 may determine that the respiratory state of the patient has improved.

Furthermore, when the second asthma symptom score is not decreased to be less than the first asthma symptom score by a predetermined score or more, the processor 110 may determine that the patient needs an outpatient appointment.

In this case, the predetermined score used by the respiratory disease patient management device 100 may be applied to various examples, such that the implementer of the inventive concept may easily select it.

Through this configuration, the device 100 or method for managing a patient with a respiratory disease according to an embodiment of the inventive concept may induce an improvement in the patient's respiratory state by determining the number of additional usages of medication depending on the symptom and state of a respiratory disease patient.

Moreover, the device 100 or method for managing a patient with a respiratory disease according to an embodiment of the inventive concept may provide a notification that an appointment for outpatient treatment is required, when the patient's respiratory state is not improved even with multiple additional doses of medication. Accordingly, the device 100 or method may accurately determine the patient's respiratory state even when the respiratory disease patient did not visit the hospital, and may allow the patient to visit a hospital only when absolutely necessary.

When a specific symptom for the corresponding patient is received from the patient terminal 50, the processor 110 may calculate an extent (hereinafter, referred to as a ‘symptom-specific risk level’) to which each symptom is dangerous, based on the first asthma symptom score calculated when the corresponding symptom is received, and the peak expiratory flow.

Also, the processor 110 may correct the first asthma symptom score by reflecting the calculated symptom-specific risk level.

This configuration uses the AI model 160, which is described in an embodiment of the inventive concept, and is a model learned based on pieces of patient data.

In an embodiment, when cause (factor) information about the patient's symptom is received from the patient terminal 50, the processor 110 may store cause information about the patient's symptom in the memory 130.

The processor 110 may derive at least one respiratory precaution for the patient based on the cause information accumulated in the memory 130.

Through this configuration, the device 100 or method for managing a patient with a respiratory disease according to an embodiment of the inventive concept may provide patient-specific respiratory precautions that each patient needs to pay special attention to.

Moreover, the processor 110 may train the individual AI model 170 by inputting the patient's symptoms stored for the individual patient, the cause (factor) information about the patient's symptoms, the peak expiratory flow received from the patient terminal 50, and the calculated first asthma symptom score as individual learning data to the AI model 160.

The device 100 or method for managing a patient with a respiratory disease according to an embodiment of the inventive concept may provide a personalized respiratory disease patient management service to each patient by using the individual AI model 170 built in this way.

In detail, when a specific symptom for the patient is received from the patient terminal 50, the processor 110 may calculate an extent (hereinafter referred to as ‘individual symptom-specific risk level’) to which the corresponding symptom is dangerous to the corresponding patient, by inputting the received specific symptom, the first asthma symptom score calculated when the symptom is received, and the peak expiratory flow to the individual AI model 170.

Also, the processor 110 may correct the first asthma symptom score by reflecting the individual symptom-specific risk level.

Through this configuration, the device 100 for managing a patient with a respiratory disease according to an embodiment of the inventive concept may provide patients with the respiratory disease patient management service by using the AI model 160 learned based on data on multiple patients, and may provide patients with the accurate respiratory disease patient management service by individually learning the AI model 160 by using the patient's personal data.

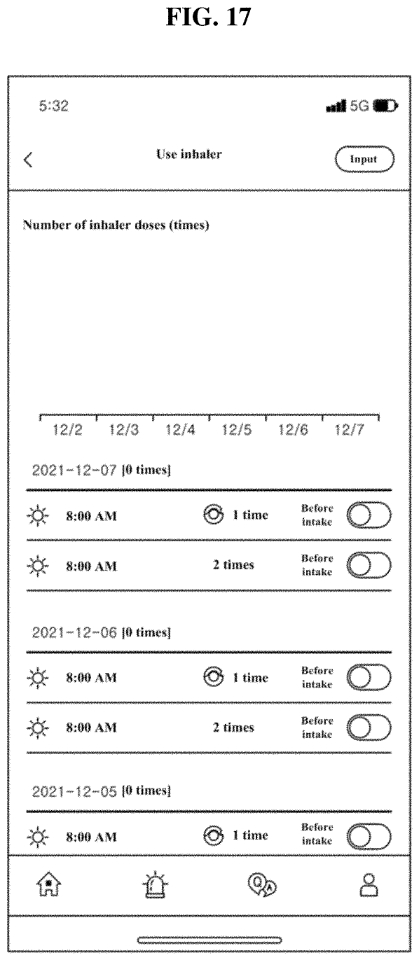

FIG. 17 is a diagram illustrating providing information about the patient's use history of the inhaler 70.

Referring to FIG. 17, the processor 110 records the number of doses of the inhaler 70 of the patient in the memory 130. When the patient terminal 50 executes a usage history inquiry function of the inhaler 70, the processor 110 may display, on the patient terminal 50, the number of times that the patient has previously taken (used) the inhaler 70, as illustrated in FIG. 17.

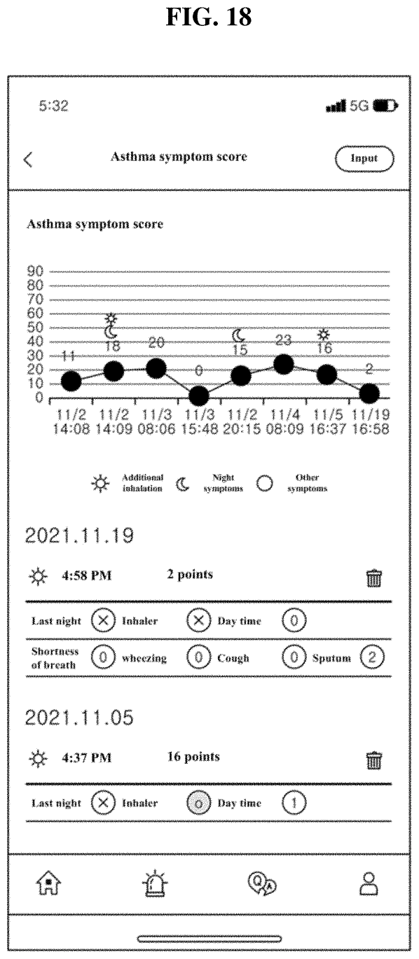

FIG. 18 is a diagram illustrating providing information about a patient's asthma symptom score history.

Referring to FIG. 18, the processor 110 may store the calculated asthma symptom score for a patient in the memory 130 together with the calculated time. When the patient terminal 50 executes an asthma symptom score inquiry function, the processor 110 may display, on the patient terminal 50, the patient's asthma symptom score history by date and time as shown in FIG. 18.

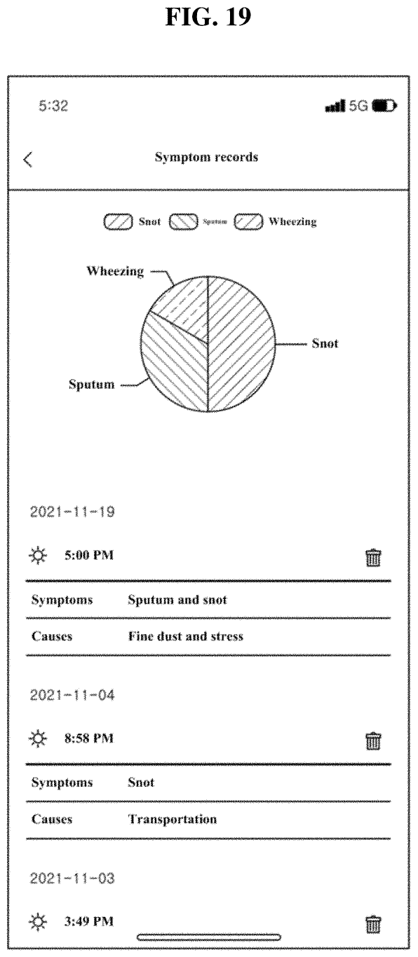

FIG. 19 is a diagram illustrating providing information about a patient's symptom record history.

Referring to FIG. 19, the processor 110 may store a symptom record of a patient in the memory 130 together with a time. When the patient terminal 50 executes the symptom record inquiry function, the processor 110 may display, on the patient terminal 50, the patient's symptom occurrence rate and occurrence time as shown in FIG. 19.

While providing functions as shown in FIGS. 17 to 19, the respiratory disease patient management device 100 provides a user interface (UI) as shown in FIG. 19 through a service application. Accordingly, patients may easily and visually identify their asthma symptom score change history through graphs and images.

The method according to an embodiment of the inventive concept may be implemented by a program (or an application) and may be stored in a medium such that the program is executed in combination with a server being hardware.

The above-described program may include a code encoded by using a computer language such as C, C++, JAVA, a machine language, or the like, which a processor (CPU) of the computer may read through the device interface of the computer, such that the computer reads the program and performs the methods implemented with the program. The code may include a functional code related to a function that defines necessary functions executing the method, and the functions may include an execution procedure related control code necessary for the processor of the computer to execute the functions in its procedures. Furthermore, the code may further include a memory reference related code on which location (address) of an internal or external memory of the computer should be referenced by the media or additional information necessary for the processor of the computer to execute the functions. Further, when the processor of the computer is required to perform communication with another computer or a server in a remote site to allow the processor of the computer to execute the functions, the code may further include a communication related code on how the processor of the computer executes communication with another computer or the server or which information or medium should be transmitted/received during communication by using a communication module of the computer.

The stored medium refers not to a medium, such as a register, a cache, or a memory, which stores data for a short time but to a medium that stores data semi-permanently and is read by a device. Specifically, for example, the stored media include, but are not limited to, ROM, RAM, CD-ROM, magnetic tape, floppy disk, optical data storage device, and the like. That is, the program may be stored in various recording media on various servers, which the computer may access, or in various recording media on the computer of the user. Further, the media may be distributed in computer systems connected over a network such that codes readable by the computer are stored in a distributed manner.

Steps or operations of the method or algorithm described with regard to an embodiment of the inventive concept may be implemented directly in hardware, may be implemented with a software module executable by hardware, or may be implemented by a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or a computer-readable recording medium well known in the art to which the inventive concept pertains.

Although an embodiment of the inventive concept are described with reference to the accompanying drawings, it will be understood by those skilled in the art to which the inventive concept pertains that the inventive concept may be carried out in other detailed forms without changing the scope and spirit or the essential features of the inventive concept. Therefore, the embodiments described above are provided by way of example in all aspects, and should be construed not to be restrictive.

According to an embodiment of the inventive concept, it is possible to provide a device for managing a patient with a respiratory disease that allows the patient with a respiratory disease to manage the respiratory disease by himself/herself.

Moreover, according to an embodiment of the inventive concept, it is possible to determine the patient's respiratory state based on the patient's peak expiratory flow and asthma symptom score.

Furthermore, according to an embodiment of the inventive concept, it is possible to provide information about the additional amount of medication required for the patient by determining the patient's respiratory state based on the patient's peak expiratory flow and asthma symptom score.

Effects of the inventive concept are not limited to the effects mentioned above, and other effects not mentioned will be clearly understood by those skilled in the art from the following description.

While the inventive concept has been described with reference to embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A respiratory disease patient management device comprising:
an artificial intelligence (AI) model configured to determine a respiratory state of a patient based on a peak expiratory flow and asthma symptom score of the patient;
a communication unit configured to communicate with a patient terminal; and
at least one processor,
wherein the processor is configured to:
receive, from the patient terminal, asthma medication intake status of the patient and the peak expiratory flow (PEF) of the patient;
calculate a first asthma symptom score of the patient based on a value entered for a plurality of asthma symptom questions provided to the patient terminal;
when at least one symptom information about the patient is received from the patient terminal, calculate first state information about the patient by inputting the received peak expiratory flow and the calculated first asthma symptom score into the AI model, and generate respiratory disease management information including whether the patient needs additional medication, based on the calculated first state information; and
transmit the generated respiratory disease management information to the patient terminal such that information for respiratory disease management of the patient is displayed on the patient terminal.

2. The respiratory disease patient management device of claim 1, wherein the AI model is configured to:
compare the received peak expiratory flow with a reference peak expiratory flow and compare the calculated first asthma symptom score with a first reference score; and
calculate the first state information about the patient based on the comparison results, and
wherein the reference peak expiratory flow and the first reference score are preset to determine additional use of medication for respiratory disease patients.

3. The respiratory disease patient management device of claim 1, wherein the processor is configured to:
derive the number of inhaler usages for additional use of medication of the patient;
provide the derived number of inhaler usages to the patient terminal; and
count the number of remaining inhaler usages based on inhalation sensing data received from the patient terminal so as to be provided to the patient terminal.

4. The respiratory disease patient management device of claim 1, wherein the processor is configured to:
allow the communication unit to load a value of a predetermined item from an EMR medical record of the patient; and
calculate the first asthma symptom score based on the loaded value.

5. The respiratory disease patient management device of claim 2, wherein the AI model is configured to:
when the received peak expiratory flow is greater than or equal to the reference peak expiratory flow, and the first asthma symptom score is increased to be greater than a first asthma symptom score on a previous test by a predetermined score or more, or when the first asthma symptom score does not exceed a first asthma symptom score on a previous test, and the received peak expiratory flow is less than the reference peak expiratory flow, determine that the patient needs one additional dose of medication.

6. The respiratory disease patient management device of claim 5, wherein the processor is configured to:
when the received peak expiratory flow is less than the reference peak expiratory flow, and the calculated first asthma symptom score is greater than or equal to the first reference score, determine that the patient needs two additional doses of medication.

7. The respiratory disease patient management device of claim 6, wherein the processor is configured to:
when the patient completes the two additional doses of medication, provide the plurality of asthma symptom questions, which are provided to the patient terminal again after a predetermined time, and calculate a second asthma symptom score of the patient based on the value entered for the plurality of asthma symptom questions thus provided again;
calculate second state information about the patient based on the calculated second asthma symptom score; and
determine whether the patient needs an outpatient appointment, based on the calculated second state information.

8. The respiratory disease patient management device of claim 7, wherein the processor is configured to:
when the second asthma symptom score is decreased to be less than the first asthma symptom score by a predetermined score or more, determine that a respiratory state of the patient is improved; and
when the second asthma symptom score is not decreased to be less than the first asthma symptom score by a predetermined score or more, determine that the patient needs an outpatient appointment.

9. The respiratory disease patient management device of claim 3, wherein the processor is configured to:
when cause information about a symptom of the patient is received from the patient terminal, store the cause information about the symptom of the patient in a memory; and
derive at least one respiratory precaution for the patient based on the cause information accumulated in the memory.

10. The respiratory disease patient management device of claim 9, wherein the processor is configured to:
store the symptom of the patient, the cause information about the symptom of the patient, the received peak expiratory flow, and the calculated first asthma symptom score in the memory;
when a specific symptom for the patient is received from the patient terminal,
calculate an extent (hereinafter, referred to as a ‘symptom-specific risk level’) to which each symptom is dangerous, based on the received specific symptom, the first asthma symptom score, which is calculated when the corresponding symptom is received, and the peak expiratory flow; and
correct the calculated first asthma symptom score by reflecting the symptom-specific risk level.

11. The respiratory disease patient management device of claim 10, wherein the processor is configured to:
train an individual AI model by inputting, as individual learning data, the symptom of the patient stored for the patient, the cause information about the symptom of the patient, the received peak expiratory flow, and the calculated first asthma symptom score.

12. The respiratory disease patient management device of claim 11, wherein the processor is configured to:
when a specific symptom for the patient is received from the patient terminal, calculate an extent (hereinafter referred to as ‘individual symptom-specific risk level’) to which the specific symptom is dangerous to the patient, by inputting the received specific symptom, the first asthma symptom score calculated when the specific symptom is received, and the peak expiratory flow to the individual AI model; and
correct the calculated first asthma symptom score by reflecting the individual symptom-specific risk level.

13. The respiratory disease patient management device of claim 1, wherein the processor is configured to:
generate a learning dataset by using at least one of the peak expiratory flow of a patient collected from a plurality of patients, an asthma symptom score of the patient, respiratory disease state information of the patient, a symptom of the patient, cause information about the symptom of the patient, the calculated first asthma symptom score, and surrounding environment information of the patient; and
perform training by inputting the generated learning dataset to the AI model.

14. The respiratory disease patient management device of claim 13, wherein the processor is configured to:
derive the surrounding environment information of the patient based on location information of the patient terminal; and
derive at least one respiratory management information based on the derived surrounding environment information by using the AI model and provide the at least one respiratory management information to the patient terminal.

15. A method performed by a respiratory disease patient management device, the method comprising:
receiving, from a patient terminal, asthma medication intake status of the patient and a peak expiratory flow (PEF) of the patient;
receiving a value entered for a plurality of asthma symptom questions provided to the patient terminal;
calculating a first asthma symptom score of the patient based on the received value;
when at least one symptom information about the patient is received from the patient terminal, calculating first state information about the patient by inputting the received peak expiratory flow and the calculated first asthma symptom score into an AI model;
generating respiratory disease management information including whether the patient needs additional medication, based on the calculated first state information; and
transmitting the generated respiratory disease management information to the patient terminal and displaying information for respiratory disease management of the patient on the patient terminal,
wherein the AI model determines a respiratory state of the patient based on the peak expiratory flow of the patient and an asthma symptom score of the patient.
